(19) United States
(12) Patent Application Publication
(10) Pub. No.: US 2009/0291156 A1
(43) Pub. Date: Nov. 26, 2009

(54) HERBAL COMPOSITION FOR SKIN CARE

(75) Inventor: Suying Liu, Belmont, CA (US)

Correspondence Address: WPAT, PC;INTELLECTUAL PROPERTY ATTORNEYS, 2030 MAIN STREET, SUITE 1300, IRVINE, CA, 92614, US

(73) Assignee: SAGITTARIUS LIFE SCIENCE CORP., Taipei Shien (TW)

(21) Appl. No.: 12/124,074

(22) Filed: May 20, 2008

(51) Int. Cl. A61K 36/254; A61Q 19/00

(52) U.S. Cl. 424/728

(57) ABSTRACT

The present invention provides an herbal composition for improving aging skin, comprising extract of Panax ginseng, Acanthopanax and Carthamus tinctorius. The composition of present invention can further comprise extract of Lignum aquilariae and Resina boswelliae carterii.

The present invention also provides a method for improving aging skin of a subject, comprising administrating a composition comprising extract of Panax ginseng, Acanthopanax and Carthamus tinctorius to the subject.

### FIELD OF THE INVENTION

This invention relates to an herbal composition for aging skin care.

This invention also relates to a method for improving aging skin of a subject.

### DESCRIPTION OF PRIOR ART

Skin is a complex organ composed of two tissues: epidermis and dermis. The epidermis, responsible for the barrier function, is a stratified and differentiated tissue. The dermis is a connective tissue with cells embedded in extracellular matrix composing of collagen, elastin, glycosaminoglycans and other macromolecules. Collagen makes up 70-80% of the dry weight of the skin and gives tensile strength to the skin. Elastin is a minor component of the dermis, and is responsible for elasticity of the skin (Emmanuelle Noblesse; et. al. J. Invest. Dermatol. 2004; 122: 621-630). Glycosaminoglycan is highly charged, and is responsible for the hydration and radiant looking of the skin among many other functions.

The clinically relevant histological changes of the skin during aging include diminished thickness of epidermis due to reduced mitosis and differentiation rate of epidermal basal layer cells, flattened rete ridges, decreased epidermal appendages, and decreased number of fibroblasts and capillaries in the dermis (Chung, J. H., et. al., J. Invest. Dermatol. 2001; 117: 1218-1224. Li L. et al., Arch. Dermatol. Res. 2006; 297: 412-416. Tagami H. Arch. Dermatol. Res. 2008; 300 (Suppl. 1): S1-S6). The structural components also changed during aging (Fisher G J., et. al., Arch. Dermatol. 2002; 138: 1462-1470). These lead to less barrier and cushion function, higher infection and trauma rate, and poor healing rate and healing quality of wounds.

Several factors contribute to the aging of the skin: genetic disorder manifestation in collagen or elastin deficiency, such as in cutis laxa; environmental excessive solar ultraviolet radiation inducing elastotic material accumulation and prematurely skin aging; and chronological aging (Robert L., et. al., Biogerontology 2000; 1: 123-131. Giacomoni P. U., et. al., Biogerontology 2001; 2: 219-229). It has been recognized that the qualitative and quantitative changes of collagen and elastin in the dermal tissues of the skin greatly affect the appearance of the skin. These protein components undergo modification by a variety of external and internal insults and in turn lead to the reduction of the elasticity, and formation of wrinkles and sag. The synthesis of type I and III collagen decreases in chronologically aged skin and is further exacerbated by photo damage which leads to failure of replacing damaged collagen. Collagen-degrading matrix metalloproteinases (MMPs) are up-regulated in skin by UV radiation and in chronological aging, and the increased MMPs is responsible for producing collagen fragmentation in skin over time (Varani J., et. al., Am. J. Pathol. 2006; 168(6): 1861-1868).

The water content and the amount of sulfated glycoaminoglycans and uronic acid of the skin decreased with age (Jung J. W., et. al., J. Dermatol. Sci. 1997; 14(1): 12-19). Glycosaminoglycans (GAGs) and proteoglycans (PGs), involved in the homeostasis and organization of dermis and other connective tissues, also shows age-related alterations (Boris Vuillermoz, et. al., Molecular and Cellular Biochemistry 2005; 277: 63-72). Heparan sulphate-proteoglycans, a subclass of the PG family, that regulate proliferation and proteolysis as well as matrix adhesion and assembly, decrease during aging. Another subclass of glycosaminoglycan, hyaluronan, is contributing to the structural and functional integrity of dermis and epidermis. The turnover of hyaluronan is perturbed in aging (Carrino D. A., et. al., Arch. Biochem. Biophys. 2000; 373(1): 91-101. Willen M. D., et. al., 1991; 96(6): 968-974).

The cross-links in collagen and elastin are essential for the tensile strength of collagens and the elastic properties of elastin, both are necessary for the structural integrity and function of connective tissues (Mäki J M., et. al., Am. J. Pathol. 2005; 167(4): 927-936). The crosslinks between collagen fibers has been considered to be one of the essential modifications during development and aging processes. Appropriate cross-linking mediated by enzymes (glutamyltransferase and lysyl oxidase) is important during development. However, the crosslinks are formed between collagen fibers non-enzymatically during aging by glycation or reactive oxygen species (Elgawish A., et. al.; J. Biol. Chem. 1996; 271(22): 12964-12971). The increased cross-links of collagen and elastin during aging affect the elasticity of skin. Collagen crosslink breakers, such as N-phenacylthiazolium (PTB), have been proposed as therapeutic agents for reversing the increase in protein crosslinking (Wolffenbuttel B. H. R., et. al., Proc. Natl. Acad. Sci. USA 1998; 95: 4630-4634).

There is increasing evidence for the generation of reactive oxygen species (ROS) in skin upon UV exposure. Increased ROS generation can overwhelm the quenching power of antioxidant defense mechanisms, and over time, cells accumulate molecular oxidative damage to lipid, protein and DNA, which may lead to an age-associated increase in photoaging of the skin. It has also been shown that exposure of human skin to UV irradiation upregulates the synthesis of the matrix-degrading enzymes matrix metalloproteinases (MMP), such as MMP-1, -2, -3, -7, -8, -9, and -12 (Sander C. S., et. al., J. Invest. Dermatol. 2002; 118: 618-625; Inomata S., et. al., J. Invest. Dermatol. 2003; 120: 128-134; Praveen K. V, et. al., J. Invest. Dermatol. 2004; 122: 1480-1487), that degrad collagen and proteoglycan.

Solar or UVA irradiation sensitizes human collagen and elastin to generate hydrogen peroxide and was reversed by antioxidant or catalase treatment (Wondrak G T., et. al., J. Invest Dermatol. 2003; 121(3): 578-586). It is known that active oxygen species, including superoxide (O₂⁻), singlet oxygen (¹O₂), hydroxyl radicals (.OH) and hydrogen peroxide (H₂O₂) attacks and cleaves glycosaminoglycan into low molecular weight fragments. Among these active oxygen species, the hydroxyl radical is considered to have a high reactivity and show the highest damaging effects. Increased oxidative stress increased MMP activity and decreased collagen synthesis (Siwik D. A., et. al., Am. J. Physiol. Cell Physiol. 2001; 280: C53-C60). It is recognized that the prevention of the generation of active oxygen and the removal of active oxygen with anti-oxidants are effective in controlling the aging of the skin, such as the formation of wrinkles and/or sags (Hu H L., et. al., Mechanisms of Ageing and Development 2000; 121: 217-230).

Cutaneous hyperpigmentation occurs in multiple conditions, such as UV activation of melanocyte, abnormality in the hormone balance, or genetic factors. UVA-induced oxidative stress is considered to promote melanogenesis and serious skin damage. Tyrosinase is the rate-limiting enzyme in melanogenesis. The commercial available whitening agents, such as L-ascorbic acid and its derivatives, hydroquinone and its derivatives, inhibit tyrosinase or suppress polymerization of 5,5-dihydroxyindole-2-carboxylic acid or prevent abnormal deposition of melanin pigments. A lighter and radiant skin tone is more desirable in many Asian women. Thus, there is a need for the development of skin lightening agents.

### BRIEF DESCRIPTION OF THE DRAWINGS

FIG. 1 shows cells treated with different herbal preparations. All the herbal preparations were at the concentration of 0.01 mg/ml in C-medium containing 25 μg/ml ascorbic acid.

A1 to A4: cells were treated with preparation CM23a;
B1 to B4: cells were treated with preparation CM23b;
C1 to C4: cells were treated with preparation CM23;
D1 to D4: cells were treated with Vitamin A (1 μM);
E1 to E4: cells were treated with PBS as control;
Lane 1, 2 were stained with Sirius red for collagen;
Lane 3, 4 were stained with Safranin O for glycosaminoglycan.

FIG. 2 shows cells treated with different herbal preparations. All the herbal preparations were at the concentration of 0.01 mg/ml in C-medium containing 25 μg/ml ascorbic acid with 0.05 mM H₂O₂.

A1 to A4: cells were treated with preparation CM23a;
B1 to B4: cells were treated with preparation CM23b;
C1 to C4: cells were treated with preparation CM23;
D1 to D4: cells were treated with Vitamin A (1 μM);
E1 to E4: cells were treated with PBS as control;
Lane 1, 2 were stained with Sirius red for collagen;
Lane 3, 4 were stained with Safranin O for glycosaminoglycan.

FIG. 3 shows Zymography of culture supernatants of dermal fibroblast treated with various herbal preparations. At molecular weights of 72K and 66K, there were the latent and active forms of MMP-2, respectively.

### Lane 1: Blank;

Lane 2: CM23a 0.02 mg/ml;
Lane 3: CM23b 0.02 mg/ml;
Lane 4: CM23 0.02 mg/ml;

### Lane 5: Control.

FIG. 4 shows Zymography of MMP in dermal fibroblast culture supernatant treated with herbal preparations in cell free conditions.

### Lane 1: Blank;

### Lane 2: Control;

### Lane 3: Control;

Lane 4: CPFa 1 mg/ml;
Lane 5: CPFa 2 mg/ml;
Lane 6: CPFb1 mg/ml;
Lane 7: CPFb 2 mg/ml;
Lane 8: CPF+1 mg/ml;
Lane 9: CPF+2 mg/ml;
Lane 10: CM23a 1 mg/ml;
Lane 11: CM23a 2 mg/ml;
Lane 12: CM23b 1 mg/ml;
Lane 13: CM23b 2 mg/ml;
Lane 14: CM23 1 mg/ml;
Lane 15: CM23 2 mg/ml;
Lane 16: CPF+1 mg/ml+CM23a 1 mg/ml;
Lane 17: CPF+1 mg/ml+CM23b 1 mg/ml;
Lane 18: CPF+1 mg/ml+CM23 1 mg/ml;

### Lane 19: Doxycycline 1 mM.

FIG. 5 shows Zymography of MMP in dermal fibroblast culture supernatant treated individually with herbal preparations in cell free conditions.

Lane 1: Molecular weight markers;

### Lane 2: Blank;

### Lane 3: Control;

Lane 4: CPFa 1 mg/ml;
Lane 5: CPFa 2 mg/ml;
Lane 6: CPFb 1 mg/ml;
Lane 7: CPFb 2 mg/ml;
Lane 8: CPF+1 mg/ml;
Lane 9: CPF+2 mg/ml;
Lane 10: CM23a 1 mg/ml;
Lane 11: CM23a 2 mg/ml;
Lane 12: CM23b 1 mg/ml;
Lane 13: CM23b 2 mg/ml;
Lane 14: CM23 1 mg/ml;
Lane 15: CM23 2 mg/ml;
Lane 16: CPF+1 mg/ml+CM23a 1 mg/ml;
Lane 17: CPF+1 mg/ml+CM23b 1 mg/ml;
Lane 18: CPF+1 mg/ml+CM23 1 mg/ml;

### Lane 19: Doxycycline 0.5 mM;

### Lane 20: Control.

FIG. 6 shows Zymography of B16F10 tyrosinase. Enzyme in gel slice was treated with the herbal preparations individually in the presence of substrate, L-DOPA. The formed melanin was at the location of tyrosinase at 66K.

### Lane 1: Blank;

### Lane 2: Control;

Lane 3: CPFa 1.0 mg/ml;
Lane 4: CPFa 2.0 mg/ml;
Lane 5: CPFb 1.0 mg/ml;
Lane 6: CPFb 2.0 mg/ml;
Lane 7: CPF+1.0 mg/ml;
Lane 8: CPF+2.0 mg/ml;
Lane 9: CM23a 1.0 mg/ml;
Lane 10: CM23a 2.0 mg/ml;
Lane 11: CM23b 1.0 mg/ml;
Lane 12: CM23b 2.0 mg/ml;
Lane 13: CM23 1.0 mg/ml;
Lane 14: CM23 2.0 mg/ml;
Lane 15: CPF+1 mg/ml+CM23a 1 mg/ml;
Lane 16: CPF+1 mg/ml+CM23b 1 mg/ml;
Lane 17: CPF+1 mg/ml+CM23 1 mg/ml;

### Lane 18: Vitamin C 0.5 mM;

### Lane 19: Control.

FIG. 7 shows Zymography of MEWO tyrosinase. Enzyme in gel slice was treated with the herbal preparations individually in the presence of substrate, L-DOPA. The formed melanin was at the location of tyrosinase at 66K.

### Lane 1: Blank;

### Lane 2: Control;

Lane 3: CPFa 1.0 mg/ml;
Lane 4: CPFa 2.0 mg/ml;
Lane 5: CPFb 1.0 mg/ml;
Lane 6: CPFb 2.0 mg/ml;
Lane 7: CPF+1.0 mg/ml;
Lane 8: CPF+2.0 mg/ml;
Lane 9: CM23a 1.0 mg/ml;
Lane 10: CM23a 2.0 mg/ml;
Lane 11: CM23b 1.0 mg/ml;
Lane 12: CM23b 2.0 mg/ml;
Lane 13: CM23 1.0 mg/ml;
Lane 14: CM23 2.0 mg/ml;
Lane 15: CPF+1 mg/ml+CM23a 1 mg/ml;
Lane 16: CPF+1 mg/ml+CM23b 1 mg/ml;
Lane 17: CPF+1 mg/ml+CM23 1 mg/ml;

### Lane 18: Vitamin C 0.5 mM;

### Lane 19: Control.

FIG. 8 shows CPF+ or vitamin C or hydroquinone treated (3 days) B16F10 cell pellets.

### Vial 1. Control;

### Vial 2. Vitamin C 0.1 mM;

### Vial 3. Hydroquinone 0.01 mM;

Vial 4. CPF+0.001 mg/ml;
Vial 5. CPF+0.003 mg/ml;
Vial 6. CPF+0.01 mg/ml;

FIG. 9 shows Zymography of B16F10 tyrosinase. B16F10 cells were treated with the herbal preparations for 3 days. Then the tyrosinase activity in cell lysate was assessed by zymography. The formed melanin was at the location of tyrosinase at 66K.

### Lane 1: Blank;

### Lane 2: Control;

### Lane 3: Vitamin C 0.1 mM;

### Lane 4: Hydroquinone 0.01 mM;

Lane 5: CPF+0.001 mg/ml;
Lane 6: CPF+0.003 mg/ml;
Lane 7: CPF+0.01 mg/ml.

FIG. 10 shows CPF+ or vitamin C or hydroquinone treated (3 days) MEWO cell pellets.

### Vial 1. Control

### Vial 2. Vitamin C 0.1 mM

### Vial 3. Hydroquinone 0.01 mM

Vial 4. CPF+0.001 mg/ml
Vial 5. CPF+0.003 mg/ml
Vial 6. CPF+0.01 mg/ml

FIG. 11 shows Zymography of MEWO tyrosinase. MEWO cells were treated with the herbal preparations for 3 days. Then the tyrosinase activity in cell lysate was assessed by zymography. The formed melanin was at the location of tyrosinase at 66K.

### Lane 1: Blank

### Lane 2: Control

### Lane 3: Vitamin C 0.1 mM

### Lane 4: Hydroquinone 0.01 mM

Lane 5: CPF+0.001 mg/ml
Lane 6: CPF+0.003 mg/ml
Lane 7: CPF+0.01 mg/ml

### SUMMARY OF THE INVENTION

The present invention provides an herbal composition for improving aging skin, comprising extract of Panax ginseng, Acanthopanax and Carthamus tinctorius.

The present invention also provides a method for improving aging skin of a subject, comprising administrating a composition comprising extract of Panax ginseng, Acanthopanax and Carthamus tinctorius to the subject.

### DETAILED DESCRIPTION OF THE INVENTION

Skin aging includes chronological aging and photo aging of the skin. The skin changes during chronological aging are accelerated by sun exposure especially UV irradiation. Examples of skin changes include dark pigmented spots (age spots); wrinkles due to changes in collagen; dryness as a result of changes in glycosaminoglycans, sweat and sebum; sagging skin owing to loss of strength and elasticity; fragile blood vessels resulting in easy bruising and bleeding, leathery skin caused by elastosis; and thinning causing less insulation and padding.

Therefore, the present invention provides a natural herbal composition with proven scientific effectiveness including stimulation of dermal fibroblast proliferation which replaces the senescence cells, stimulation of collagen synthesis which enhances firmness and has anti-wrinkle effect, stimulation of glycosaminoglycan synthesis which increases hydration and radiance of the skin, anti-oxidation of ROS (DPPH radical scavenging), anti UV-induced MMP effect which provides anti-wrinkle function, anti-tyrosinase mediated melanogenesis effect which result in whitening and depigmentation. Therefore, the present invention acts on a whole spectrum of essential factors related to skin aging.

More specifically, the present invention provides an herbal composition for improving aging skin, comprising extract of Panax ginseng, Acanthopanax and Carthamus tinctorius.

In a preferred embodiment, the composition of present invention further comprises extract of Lignum aquilariae and Resina boswelliae carterii.

Preferably, the extract of Panax ginseng, Acanthopanax, Carthamus tinctorius has a weight-to-weight ratio of 0.1-1:0.1-1:0.03-0.3 and the extract of Lignum aquilariae and Resina boswelliae carterii has a weight-to-weight ratio of 0.3-3:0.3-3.

In another preferred embodiment, the composition of present invention further comprises pharmaceutically or cosmetically acceptable excipient, diluent, or carrier.

Preferably, the excipient, diluent, or carrier can be formulated, but not limited, into cream, lotion, plasma, spray, liquid, tablet, capsule, granule, powder or solution dosage forms.

The present invention also provides a method for improving aging skin of a subject, comprising administrating a composition comprising extract of Panax ginseng, Acanthopanax and Carthamus tinctorius to the subject.

In a preferred embodiment, the composition is further comprising extract of Lignum aquilariae and Resina boswelliae carterii.

In a preferred embodiment, the composition is administrated by topical, transdermal, oral, intracutaneous injection or subcutaneous injection means.

In a preferred embodiment, the method stimulates a reaction selected from the group consisting of fibroblast proliferation, collagen synthesis and glycosaminoglycan synthesis.

In another preferred embodiment, the method inhibits MMP-2 activity under peroxide or UV irradiation and inhibits a reaction selected from the group consisting of tyrosinase activity and melanin formation.

In the other preferred embodiment, the method has an anti-oxidation activity.

In a preferred embodiment, the method comprising administrating a composition comprising extract of Panax ginseng, Acanthopanax and Carthamus tinctorius and extract of Lignum aquilariae and Resina boswelliae carterii to the subject has an anti-aging effect selected from the group consisting of senescent dermal fibroblast cell replacement, anti-wrinkle, skin hydration, depigmentation and whitening.

In another embodiment, said method increases a function selected from the group consisting of firmness, hydration and radiance in aging skin of a subject.

The present invention further provides an herbal composition for improving aging skin, comprising extract of Lignum aquilariae and Resina boswelliae carterii.

Preferably, the extract of Lignum aquilariae and Resina boswelliae carterii has a weight-to-weight ratio of 0.3-3:0.3-3.

In a preferred embodiment, the composition further comprises pharmaceutically or cosmetically acceptable excipient, diluent, or carrier.

Preferably, the excipient diluent, or carrier can be formulated, but not limited, into cream, lotion, plasma, spray, liquid, tablet, capsule, granule, powder or solution dosage forms.

The present invention also provides a method for improving aging skin of a subject, comprising administrating a composition comprising extract of Lignum aquilariae and Resina boswelliae carterii.

Preferably, the composition is administrated by topical, transdermal, oral, intracutaneous injection or subcutaneous injection means.

In a preferred embodiment, the method stimulates a reaction selected from the group consisting of fibroblast proliferation, collagen synthesis, glycosaminoglycan synthesis, tyrosinase inhibition, MMP inhibition and anti-oxidation.

In another preferred embodiment, the method has an anti-aging effect selected from the group consisting of senescent dermal fibroblast cell replacement and anti-wrinkle, and whitening.

In the other preferred embodiment, the method increases a function selected from the group consisting of firmness, hydration, radiance and depigmentation in aging skin of a subject.

The examples below are non-limiting and are merely representative of various aspects and features of the present invention.

### EXAMPLE

### Chemistry

### Example 1

### Composition and Manufacture Processes

### Composition of CPF+

CPF+ comprises of Panax ginseng, Acanthopanax senticosus, Carthamus tinctorius, at the weight-to-weight ratio of 0.1-1:0.1-1:0.03-0.3.

### Manufacture Processes of CPF+

A. Carthamus tinctorius was extracted with 5 to 10 folds of water by refluxing at 100±5° C. for one hour. The supernatant was filtered by passing through 400 mesh filter. This supernatant was concentrated and dried, and was designated CPFa.

B. Panax ginseng and Acanthopanax senticosus were extracted with 5 to 10 folds of 50±10% aqueous ethanol by refluxing at 100±5° C. for one hour twice. The pooled supernatant was filtered by passing through 400 mesh filter. This supernatant was concentrated and dried, and was designated CPFb.

C. Supernatant in A and supernatant in B were mixed, concentrated, and dried. It was designated CPF+.

### Composition of CM23

CM23 comprises of Lignum aquilariae and Resina boswelliae carterii at the weight-to-weight ratio of 0.3-3:0.3-3.

### Manufacture Processes of CM23

Lignum aquilariae and Resina boswelliae carterii were extracted with 5 to 10 folds of water or 50±10% ethanol or 95% ethanol by refluxing at 100±5° C. for one hour twice. The supernatants were pooled and filtered through 400 mesh filter. The supernatant was concentrated and dried, and was designated CM23a (water extract), CM23b (50% ethanol extract), CM23 (95% ethanol extract), respectively.

### Biological Activities

### Materials

CPFa comprises Carthamus tinctorius.

CPFb comprises Panax ginseng and Acanthopanax senticosus.

CPF+ comprises Panax ginseng, Acanthopanax senticosus and Carthamus tinctorius.

CM23 comprises Lignum aquilariae and Resina boswelliae carterii.

### Example 2

### Stimulation of Dermal Fibroblast Proliferation

Human foreskin dermal fibroblasts were cultured in DMEM+10% FCS+Penicillin G (100 unit/ml)/Streptomycin (100 ug/ml)+Glutamine (2 mM)+Sodium pyruvate (0.1 mM) (C-medium) under humidifying atmosphere containing 5% CO₂ at 37° C.

1×10⁴ cells were plated into each well of 96 multiwell plate. 24 hours later, the medium was replaced with fresh C-medium containing various concentrations of herbal preparations. After treatment for 4 days, the media were replaced with MTT at 0.5 mg/ml in PBS and incubated under humidifying atmosphere containing 5% CO₂ at 37° C. for 4 hours. At the end of incubation, MTT solution was aspirated and the reduced formasan precipitate was dissolved by isopropanol containing 0.04 N HCl, and read at 570 nm by an ELISA reader (Molecular Device).

TABLE 1
Stimulation of dermal fibroblast proliferation by CM23 and CPF+

| | CM23 | | CPF+ | |
|---|---|---|---|---|
| Conc. (mg/ml) | Mean OD | % change | Mean OD | % change |
| 0.1 | 0.036 | −100 | 0.209 | −33 |
| 0.03 | 0.334 | 17 | 0.327 | 14 |
| 0.01 | 0.309 | 7 | 0.307 | 6 |
| 0.003 | 0.271 | −8 | 0.289 | −1 |
| 0 | 0.291 | | 0.292 | |
| Blank | 0.040 | | 0.040 | |

CM23 at 0.1 mg/ml was toxic to the cells, at 0.03, or 0.01 mg/ml were mildly stimulatory to fibroblast proliferation. CPF+ showed similar effects at the same concentration ranges.

### Example 3

### Stimulation of Dermal Fibroblast Collagen and Glycosaminoglycan Synthesis

5×10⁴ cells were plated into each well of 48 multiwell plate. Three to four days later, the cells reached confluency. The medium was replaced with fresh C-medium with or without H₂O₂ containing 25 ug/ml vitamin C with herbal preparations, Genistein or vitamin A. After treatment for 7 days, the cells were fixed with 10% formalin in saline, and stained with Sirius red, Safranin O or Alcian blue (at pH2.5 for hyaluronic acid staining). The relative amount of extracellular matrix deposition was estimated by visual comparison of the intensity and area of the respective stains.

A. Stimulation of Dermal Fibroblast Collagen and Glycosaminoglycan Synthesis by Herbal Preparations Under the Cultured Conditions without H₂O₂, Compared with Genistein

TABLE 2
Stimulation of dermal fibroblast collagen and glycosaminoglycan synthesis by herbal
preparations (0.01 mg/ml) under the cultured conditions without H₂O₂

| | CPFa | CPFb | CPF+ | CM23a | CM23b | CM23 | Genistein | Control |
|---|---|---|---|---|---|---|---|---|
| Sirius Red | ++ | ++ | +++ | ++ | +++ | ++++ | ++ | ++ |
| Safranin O | ++ | ++ | +++ | ++ | +++ | ++++ | ++ | ++ |
| Alcian Blue | +++ | +++ | +++ | ++ | +++ | ++++ | ++ | ++ |

As shown in Table 2, CM23 demonstrated the highest stimulatory effects of collagen and glycosaminoglycan synthesis, followed by CPF+, while Genistein at 10 uM did not show significant effect under the same experimental conditions.

B. Stimulation of Dermal Fibroblast Collagen and Glycosaminoglycan Synthesis by Herbal Preparations in the Presence of 0.1 Mm H₂O₂ Compared with Genistein

TABLE 3
Stimulation of dermal fibroblast collagen and glycosaminoglycan synthesis by
herbal preparations (0.01 mg/ml) in the presence of 0.1 mM H₂O₂

| | CPFa | CPFb | CPF+ | CM23a | CM23b | CM23 | Genistein | Control |
|---|---|---|---|---|---|---|---|---|
| Sirius Red | + | + | ++ | + | ++ | +++ | + | + |
| Safranin O | + | + | ++ | + | ++ | +++ | + | + |
| Alcian Blue | ++ | ++ | + | + | + | +++ | + | + |

As shown in Table 3, CM23 demonstrated the highest stimulatory effects of collagen and glycosaminoglycan synthesis, followed by CM23b and CPF+, while Genistein at 10 μM did not show significant effect in the presence of 0.1 mM H₂O₂.

C. Stimulation of Dermal Fibroblast Collagen and Glycosaminoglycan Synthesis by Herbal Preparations Under the Cultured Conditions without H₂O₂, Compared with Vitamin A

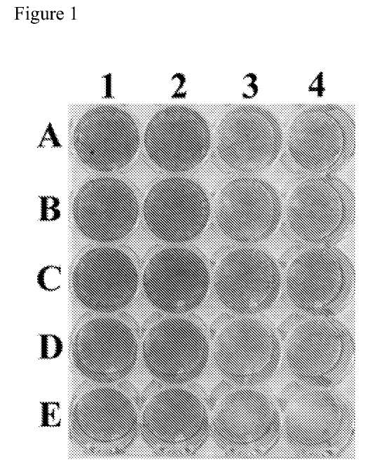

As shown in FIG. 1, CM23 demonstrated the highest stimulatory effect on collagen and glycosaminoglycan synthesis, while Vitamin A at 1 μM did not show significant activity under the same experimental conditions compared to control.

D. Stimulation of Dermal Fibroblast Collagen and Glycosaminoglycan Synthesis by Herbal Preparations in the Presence of 0.05 Mm H₂O₂, Compared with Vitamin A

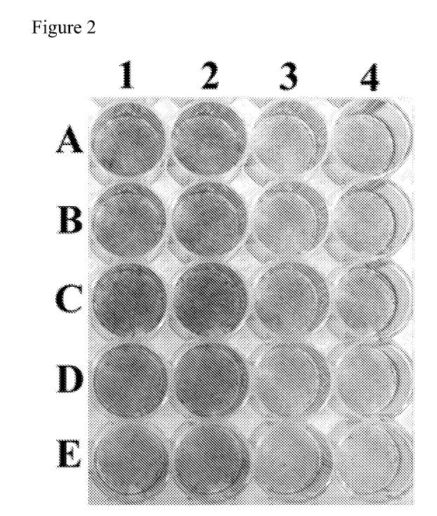

As shown in FIG. 2, CM23 demonstrated the highest stimulatory effect on collagen and glycosaminoglycan synthesis in the presence of 0.05 mM H₂O₂, while Vitamin A at 1 μM did not show significant activity under the same experimental conditions.

E. Stimulation of Dermal Fibroblast Collagen and Glycosaminoglycan Synthesis by Various Concentrations of Herbal Preparations Compared with Vitamin A

TABLE 4
Stimulation of dermal fibroblast collagen and glycosaminoglycan
synthesis by various concentrations of herbal preparations

| | | | | | | | Vit A | |
|---|---|---|---|---|---|---|---|---|
| | CPF+ (mg/ml) | | | CM23 (mg/ml) | | | (μM) | Control |
| | 0.01 | 0.02 | 0.04 | 0.01 | 0.02 | 0.04 | 10 | — |
| Sirius Red | ++ | ++ | +++ | +++ | +++ | ++++ | + | ++ |
| Safranin O | ++ | ++ | +++ | + | + | ++ | + | ++ |
| Alcian Blue | +++ | +++ | ++++ | + | + | + | + | ++ |

As shown in Table 4, CPF+ and CM23 stimulated collagen, sulfated glycosaminoglycan, non-sulfated glycosaminoglycan (hyaluronic acid) synthesis dose-dependently. CPF+ was more potent than CM23 on the stimulation of hyaluronic acid synthesis. Vitamin A at 10 μM did not show significant effect.

F. Stimulation of Dermal Fibroblast Collagen and Glycosaminoglycan Synthesis by Various Concentrations of Herbal Preparations with UV Irradiation Compared with Vitamin A

At the end of treatments, the cells were washed twice with PBS, and were irradiated by UV (365 nm) at 20 mJ/cm² in PBS. Fresh serum free medium was replaced, and the cells were fixed and stained 24 hours later.

TABLE 5
Stimulation of dermal fibroblast collagen and glycosaminoglycan
synthesis by various concentrations of herbal preparations and
protecting from UV trauma (365 nm at 20 mJ/cm² in PBS)

| | | | | | | | Vit A | |
|---|---|---|---|---|---|---|---|---|
| | CPF + (mg/ml) | | | CM23 (mg/ml) | | | (μM) | |
| | Conc. | | | | | | | |
| | 0.01 | 0.02 | 0.04 | 0.01 | 0.02 | 0.04 | 10 | Control |
| Sirius Red | ++ | ++++ | ++++ | +++ | +++ | ++++ | + | ++ |
| Safranin O | ++ | +++ | +++ | ++ | ++ | ++ | + | ++ |
| Alcian Blue | +++ | +++ | ++++ | ++ | ++ | ++ | + | ++ |

As shown in Table 5, CPF+ and CM23 stimulated collagen, sulfated glycosaminoglycan, non-sulfated glycosaminoglycan (hyaluronic acid) dose-dependently. Vitamin A at 10 μM did not show significant effect. UV irradiation induced MMP activity which degraded extracellular matrix. Compared to the treatment in Table 4 that was not UV irradiated, the depositions of collagen glycosaminoglycan, hyaluronic acid was slightly less. UV irradiation did not appear to inhibit the effects of these herbal preparations on the stimulation of collagen and glycosaminoglycan synthesis.

### Example 4

### MMP Activity in Human Dermal Fibroblasts after Treatment of Herbal Preparations

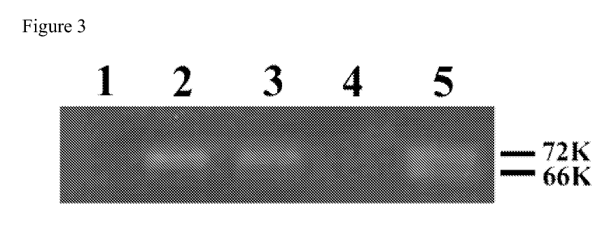

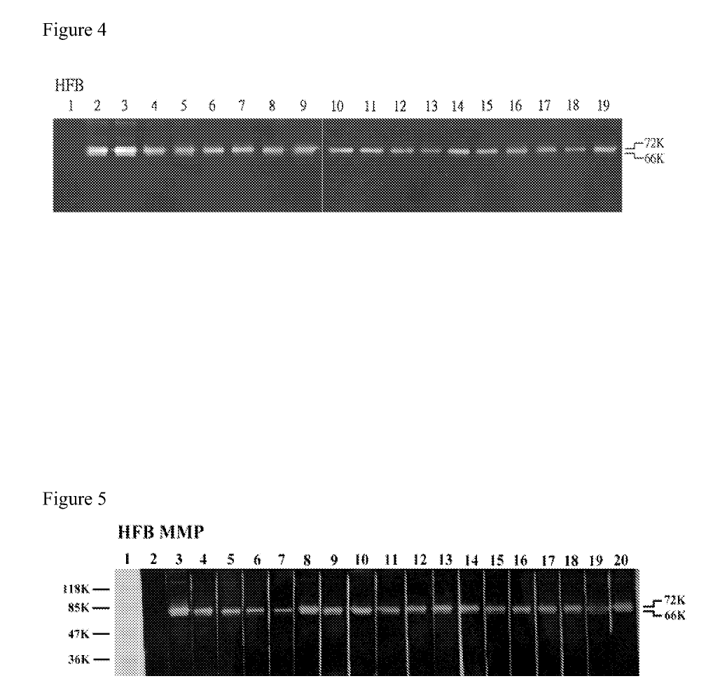

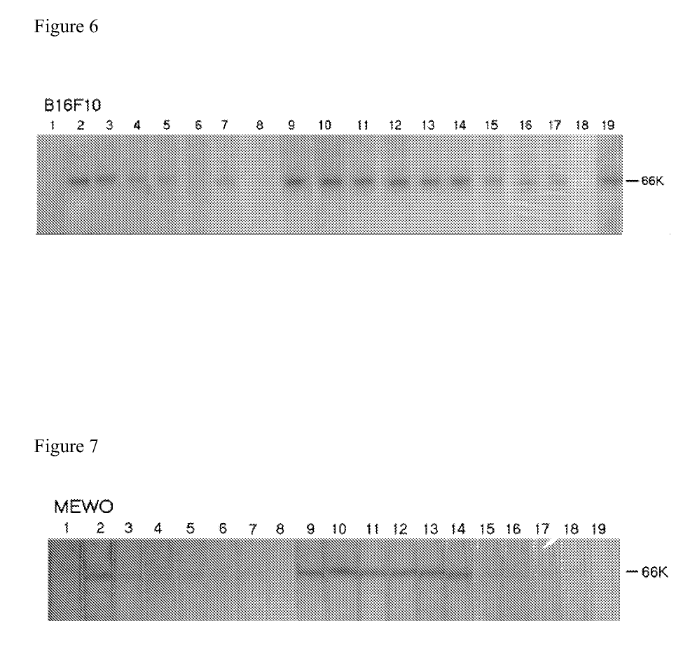

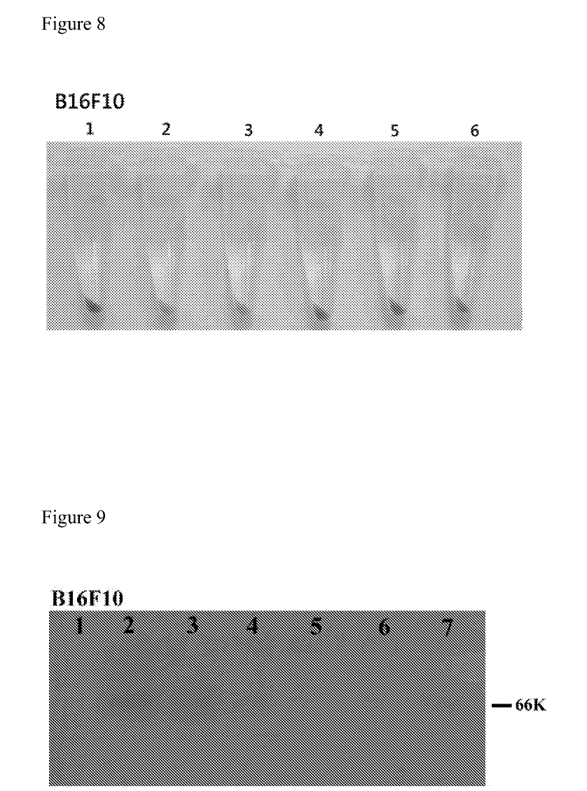

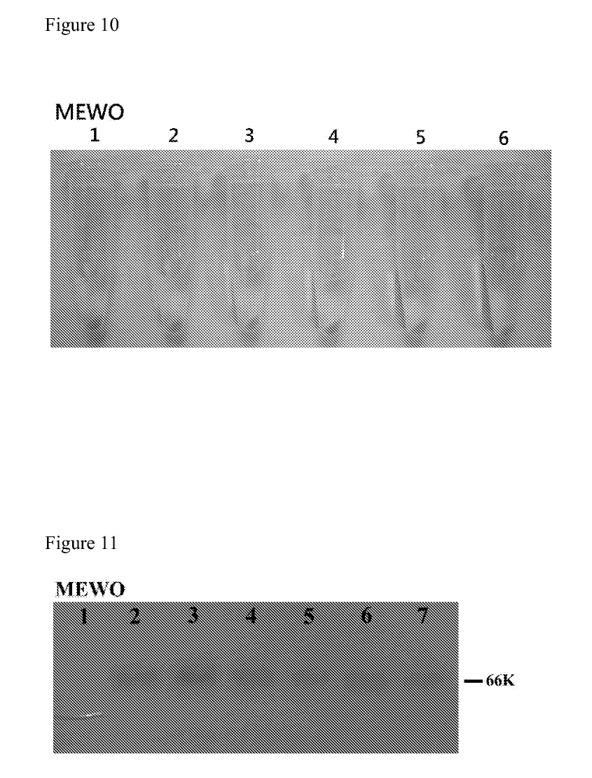

5×10⁴ cells were plated into each well of 48 well plate. Two to 3 days later, the cells reached confluency. The medium was replaced with fresh C-medium containing 25 μg/ml vitamin C with 0.02 mg/ml of herbal preparations. After treatment for 7 days, the cells were washed with PBS, and incubated for 24 hours in DMEM. The media were collected and protein concentrations were determined by BioRad protein assay solution using bovine serum albumin as standard. Equal amounts of protein in SDS sample buffer without reducing agent were loaded into each well of 10% SDS polyacrylamide gel containing 0.1% gelatin. The gel was run at 90 volts by BioRad Mini Protean gel electrophoresis apparatus. The gel was washed three times with 2.5% Triton X-100 to replace SDS, 15 minutes each, and then developed with developing buffer (50 mM Tris-HCl, pH 7.6, 10 mM CaCl₂, 50 mM NaCl, 0.05% Brij 35) at 37° C. for 24 hours. The gel was then stained with 0.1% Coomassie blue R250 in 40% methanol and 10% acetic acid. The MMP-2 (gelatinase) was visualized as clear bands in blue background after destained in 30% methanol and 7% acetic acid. As shown in FIG. 3, both of the latent form and active form of MMP-2 were inhibited by CM23. The active form was inhibited by CM23a, CM23b.

### Example 5

### MMP Activity in Culture Supernatant of Human Dermal Fibroblasts After Treatment of Herbal Preparations Under Cell Free Condition

The confluent human dermal fibroblast was cultured in DMEM under humidifying atmosphere containing 5% CO₂ at 37° C. for 24 hours. The culture supernatant was collected.

Equal amounts the culture supernatant (10 μl) were incubated with 10 μl of herbal preparations (final concentration: 0.01 and 0.1 mg/ml) in 0.1 M phosphate buffer at pH6.8 at 37° C. for 1 hour. Equal volumes (20 μl) of 2×SDS sample buffer without reducing agent were added into the reaction mixture to stop the reaction. The samples were set at room temperature for two hours and equal volume of the samples was loaded into each well of 10% SDS polyacrylamide gel containing 0.1% gelatin. The gel was run at 90 volts by BioRad Mini Protean gel electrophoresis apparatus. The gel was washed three times with 2.5% Triton X-100 to replace SDS, 15 minutes each, and then developed with developing buffer (50 mM Tris-HCl, pH 7.6, 10 mM CaCl₂, 50 mM NaCl, 0.05% Brij 35) at 37° C. for 24 hours. The gel was then stained with 0.1% Coomassie blue R250 in 40% methanol and 10% acetic acid. The MMP-2 (gelatinase) was visualized as clear bands in blue background after destained in 30% methanol and 7% acetic acid.

As shown in FIG. 4, CPF+ series moderately inhibited MMP-2 dose dependently, while CM23 series was more potent on MMP-2 inhibition than CPF+ series. CM23b was the most potent inhibitor in this cell free system. Combination of CPF+ with CM23 series appeared to maintain relatively similar MMP inhibition activity of CM23 series, and CPF+ combined with CM23 showed the most potent inhibition. The positive control, doxycycline at 1 mM showed about 50% inhibition of MMP-2 activity under this experimental condition.

### Example 6

### Inhibition of MMP Activity in Culture Supernatant of Human Dermal Fibroblasts by Herbal Preparations Individually Under Cell Free Condition

Equal volume of dermal fibroblast culture supernatant was treated with 2×SDS sample buffer without reducing agent at room temperature overnight. Equal volumes of the denatured sample were loaded onto each well of 10% SDS polyacrylamide gel containing 0.1% gelatin, and was electrophoresized at 90 volts. After the dye front reached the bottom of the gel, the gel was washed 3 times with 2.5% Triton X-100, 15 minutes each. The gel was then sliced and incubated individually with various herbal preparations in developing buffer (50 mM Tris-HCl, pH 7.6, 10 mM CaCl₂, 50 mM NaCl, 0.05% Brij 35) at 37° C. for 24 hours. The gel slice was then stained with 0.1% Coomassie brilliant blue R250 in 40% methanol and 10% acetic acid. The MMP-2 (gelatinase) was visualized as clear bands in blue background after destained in 30% methanol and 7% acetic acid.

As shown in FIG. 5, CM23 series inhibited MMP-2 dose dependently, while CPF+ series was more potent on MMP-2 inhibition than CM23 series. CPFb was the most potent inhibitor in this cell free system. Combination of CM23 series with CPF+ appeared to relatively blocked MMP inhibition activity of CPF+. The positive control, doxycycline at 0.5 mM showed about 50% inhibition of MMP-2 activity under this experimental condition.

### Example 7

### Tyrosinase Activity and Melanin Formation in Cell Free System

Mouse B16F10 or human MEWO human melanoma cells were grown in DMEM+5% FCS+Penicillin G (100 unit/ml)/Streptomycin (100 ug/ml)+2 mM glutamine+Na pyruvate (0.1 mM) (C-medium) under humidifying atmosphere containing 5% CO₂ at 37° C. Confluent cells were collected by trypsinization, washed with PBS and lyzed in mammalian cell lysis solution (Cellyte, Sigma). The cell lysate was freeze-thaw three times, and centrifuged at 13,000 rpm for 5 minutes. The supernatant was collected and assayed for protein concentration. Twenty ug of protein in SDS sample buffer without reducing agent were loaded into each well of 10% SDS polyacrylamide gel. The gel was run at 90 volts by BioRad Mini Protean gel electrophoresis apparatus. After the dye front reached the bottom of the gel, the gel was washed and renatured in 0.1 M phosphate buffer, pH6.8, twice, each for 30 minutes. The sliced gels were then individually incubated with inhibitors of tyrosinase in 0.1 M phosphate buffer, pH6.8 containing 0.5 mM L-DOPA at 37° C. overnight. The formed melanin was visualized as black band in the gel slices.

As shown in FIG. 6, CPF+ series dose-dependently inhibited tyrosinase, CPFb and CPF+ showed comparable inhibitory activities. Under the same experimental conditions, CM23 series did not show inhibitory activity on tyrosinase. Combination of CPF+ with CM23 series appeared to decrease the potency of tyrosinase inhibition of this mouse cell enzyme. As shown in FIG. 7, CPF+ series dose-dependently inhibited tyrosinase, CPFb and CPF+showed comparable inhibitory activities. Under the same experimental conditions, CM23 series did not show inhibitory activity on tyrosinase. Combination of CPF+ with CM23 series did not appear to decrease the potency of tyrosinase inhibition of this human cell enzyme.

### Example 8

### Inhibition of Tyrosinase Activity and Melanin Formation in B16F10 Melanoma or MEWO Melanoma Cells

2.5×10⁵B16F10 or MEWO melanoma cells were grown in 6 well plate in DMEM+5% FCS+Penicillin G (100 unit/ml)/streptomycin (100 ug/ml)+2 mM glutamine+Na pyruvate (0.1 mM) under humidifying atmosphere containing 5% CO₂ at 37° C. After the cells reached confluency, the cells were treated with various herbal preparations in DMEM containing 2% FCS for 3 days. At the end of treatment, the cells were washed with PBS, trypsinized, counted, pelleted and lyzed in mammalian cell lysis solution (CelLytic™ M, Sigma; 1×10⁷/125 μl). After freeze/thaw three times, the supernatant of cell lysate was collected by centrifuged at 13,000 rpm for 15 minutes at 4° C., and assayed for protein concentration. Twenty ug of protein in SDS sample buffer without reducing agent were loaded into each well of 10% SDS polyacrylamide gel. The gel was run at 90 volts by BioRad Mini Protean gel electrophoresis apparatus. After the dye front reached the bottom of the gel, the gel was washed and renatured in 0.1 M phosphate buffer, pH6.8, twice, each for 30 minutes. The gels were then each incubated in 0.1 M phosphate buffer, pH6.8 containing 0.5 mM L-DOPA at 37° C. overnight. The formed melanin was visualized as black band in the gel.

As shown in FIG. 8, CPF+ dose-dependently inhibited melanin formation. At 0.01 mg/ml, it showed comparable activity as hydroquinone at 0.01 mM.

As shown in FIG. 9, CPF+ dose-dependently inhibited tyrosinase activity. At 0.01 mg/ml, it completely inhibited tyrosinase activity. At 0.001 mg/ml, it showed comparable activity as hydroquinone at 0.01 mM.

As shown in FIG. 10, CPF+ dose-dependently inhibited melanin formation. At 0.01 mg/ml, it showed comparable activity as hydroquinone at 0.01 mM.

As shown in FIG. 11, CPF+ dose-dependently inhibited tyrosinase activity. At 0.001 mg/ml, it showed more potent inhibitory activity than vitamin C at 0.1 mM as well as hydroquinone at 0.01 mM.

### Example 9

### The Radical Scavenging Activity of CPF+ and CM23 Examined by DPPH

Herbal preparations with the designated final concentrations was mixed with 0.1 mM DPPH (2,2-diphenyl-1-picrylhydrazyl), incubated at room temperature in dark for 30 minutes. Absorbance at 517 nm was read by Varian Cary 50 Tablet UV-Visible Spectrophotometer. Vitamin C and vitamin E served as positive controls.

TABLE 6
Radical scavenging activity of the herbal preparations

| | % DPPH radical scavenging | | | | | | | | | |
|---|---|---|---|---|---|---|---|---|---|---|
| Conc. | | | | | | | Conc. | | Conc. | |
| (mg/ml) | CPFa | CPFb | CPF+ | CM23a | CM23b | CM23 | (mM) | Vit. C | (mM) | Vit. E |
| 0.01 | 1.4 | 4.9 | 20.8 | 4.9 | 6.9 | 6.9 | 0.001 | 75.0 | 0.01 | 70.1 |
| 0.1 | 18.1 | 20.1 | 71.5 | 6.9 | 7.6 | 4.9 | 0.01 | 99.3 | 0.1 | 86.8 |

At 0.1 mg/ml, CPF+ scavenged 71.5% of radical as assayed by DPPH, while Vitamin C at 0.001 mM and Vitamin E at 0.01 mM showed similar potency.

### Example 10

### Human Use Experience

TABLE 7
Plasma

| Ingredient | Content (%) |
|---|---|
| Butylene glycol | 7.00 |
| Triethylhexanoin | 5.00 |
| Glycerin | 5.00 |
| Butyl glycol | 5.00 |
| Polyquaternium-39 | 2.00 |
| Acrylates/C10-30 alkyl acrylate | 0.40 |
| crosspolymer | |
| Carbomer | 0.10 |
| Polysorbate-20 | 0.10 |
| Sodium hydroxide | 0.05 |
| Sodium hyaluronate | 0.05 |
| Methylisothiazolinone | 0.03 |
| Benzisothiazolinone | 0.02 |
| Fragrance | 0.10 |
| CPF+ | 0.362 |
| CM23 | 0.438 |
| Water | 74.35 |
| Total | 100.00 |

Ten female, age 25 to 50, participated in the trial use of the plasma containing the present invention. They were instructed to practice their normal skin care except not to use any material after cleaning the face in the evening. After one week run-in washout, the plasma was applied to the skin of the face after thorough cleaning in the evening. After 6 weeks of application, the global self-assessment of facial skin improved 50% in 50% of the participants. After 12 weeks of application, the global self-assessment of facial skin improved 75% in 90% of the participants. Assessment items included firmness, hydration, wrinkles, radiance and whitening (score 1 to 5 for each category, the total scores of the 5 categories at week 6 and week 12 were compared with baseline).

Summaries shown in Table 1, CM23 and CPF+ at 0.03, 0.01 mg/ml were stimulatory to fibroblast proliferation. The percentage of stimulation was at the range of replenishing the loss of fibroblast during aging process. These two preparations also demonstrated stimulatory effects of collagen and glycosaminoglycan synthesis (Table 2 to 5; FIG. 1 to 2). In the presence of 0.1 mM H₂O₂, CPF+ and CM23 retained their stimulatory effects on collagen and glycosaminoglycan synthesis, while anti-oxidants, Genistein, at 10 uM in the presence of 0.1 mM H₂O₂, vitamin A at 1 uM in the presence of 0.05 mM H₂O₂ did not show stimulatory effect on collagen and glycosaminoglycan synthesis. Under UV irradiation, CM23 and CPF+ were still able to stimulate collagen and glycosaminoglycan deposition dose dependently. It remained active under the stress of UV irradiation or H₂O₂ peroxidation.

As shown in FIG. 3, CM23a, CM23b, and CM23 inhibited both of the latent form and active form of MMP-2 in dermal fibroblasts. CM23 was the most potent one. In cell free system (FIGS. 4 and 5), CM23b and CM23 had similar activity on inhibition of MMP-2. In FIG. 4, CPF+might have been separated from enzyme complex during electrophoresis; therefore, it did not show potent inhibitory activity when the substrate was present.

As shown in FIGS. 6 and 7 in cell free system, CPF at 1 mg/ml were the most potent one on inhibiting tyrosinase and melanin formation of B16F10 and MEWO tyrosinase, respectively. When cells were treated with CPF+ as shown in FIG. 8 to 11, melanin formation and tyrosinase activity in B10F10 and MEWO melanoma cells were inhibited dose-dependently. Under the same experimental conditions, ascorbic acid was the most potent one. However, ascorbic acid is very unstable. It is very difficult to formulate it to retain its activity for cosmetic application. CPF+ also showed anti-oxidation activity as assayed by DPPH radical scavenging activity (Table 6).

The stimulation of dermal fibroblast proliferation and differentiation (production of collagen and glycosaminoglycan) and inhibition of MMP-2 by CM23 and CPF+ under peroxide and UV trauma render these two preparations potential to provide tensile strength and firmness (by collagen) of the photo-aging and chronological aging skin, in other words, to reduce wrinkle formation and retain water content (by highly negative charged glycosaminoglycan) of the skin. CPF+ inhibited tyrosinase activity and melanin formation as shown in FIG. 6 to 11. In addition, CPF+ also had anti-oxidation activity. Combination of CM23 which had high stimulatory activity on collagen and glycosaminoglycan synthesis and deposition, with CPF+ which had high tyrosinase inhibition and anti-oxidation activities rendered the present invention a novel herbal composition simultaneously combat on all the factors in aging skin: wrinkle to smoothness, sag to firmness, flaky dry to hydration, dull to radiance, and pigmentation/melasma to whitening. It had been proved by human use experience of the plasma of Table 7 in Example 10.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

What is claimed is:

1. An herbal composition for improving aging skin, comprising extract of Panax ginseng, Acanthopanax and Carthamus tinctorius.

2. The composition of claim 1 further comprises extract of Lignum aquilariae and Resina boswelliae carterii.

3. The composition of claim 1, wherein the extract of Panax ginseng, Acanthopanax, Carthamus tinctorius has a weight-to-weight ratio of 0.1-1:0.1-1:0.03-0.3.

4. The composition of claim 2, wherein the extract of Lignum aquilariae and Resina boswelliae carterii has a weight-to-weight ratio of 0.3-3:0.3-3.

5. The composition of claim 1, which further comprises pharmaceutically or cosmetically acceptable excipient, diluent, or carrier.

6. The composition of claim 1, wherein the excipient, diluent, or carrier is formulated into cream, lotion, plasma, spray, liquid, tablet, capsule, granule, powder or solution dosage forms.

7. A method for improving aging skin of a subject, comprising administrating a composition comprising extract of Panax ginseng, Acanthopanax and Carthamus tinctorius to the subject.

8. The composition of claim 1, wherein the composition further comprising extract of Lignum aquilariae and Resina boswelliae carterii.

9. The method of claim 13, wherein the composition is administrated by topical, transdermal, oral, intracutaneous injection or subcutaneous injection means.

10. The method of claim 7, which stimulates a reaction selected from the group consisting of fibroblast proliferation, collagen synthesis and glycosaminoglycan synthesis.

11. The method of claim 7, which inhibits MMP-2 activity under peroxide or UV irradiation.

12. The method of claim 7, which inhibits a reaction selected from the group consisting of tyrosinase activity and melanin formation.

13. The method of claim 7, which has an anti-oxidation activity.

14. The method of claim 8, which has an anti-aging effect selected from the group consisting of senescent dermal fibroblast cell replacement, anti-wrinkle, skin hydration, depigmentation and whitening.

15. The method of claim 8, which increases a function selected from the group consisting of firmness, hydration and radiance in aging skin of a subject.
